(19) United States
(12) Patent Application Publication
(10) Pub. No.: US 2020/0022777 A1
(43) Pub. Date: Jan. 23, 2020

(54) SYSTEMS, APPARATUSES, AND METHODS FOR CLEARING DIALYSIS CATHETERS

(71) Applicant: Kumara RAMA, St. Clair Shores, MI (US)

(72) Inventor: Kumara RAMA, St. Clair Shores, MI (US)

(21) Appl. No.: 16/039,164

(22) Filed: Jul. 18, 2018

(51) Int. Cl. A61B 90/70; B08B 9/043

(52) U.S. Cl. CPC A61B 90/70; A61M 2025/0019; A61B 2090/701; B08B 9/0436

(57) ABSTRACT

The disclosure generally relates to systems, apparatuses or devices, and methods for: conditioning surfaces of dialysis catheters; removing or clearing occlusions within dialysis catheters; or collecting tissue or cell samples.

### TECHNICAL FIELD

The disclosure generally relates to systems, apparatuses or devices, and methods for: conditioning surfaces of dialysis catheters; removing or clearing occlusions within dialysis catheters; or collecting tissue or cell samples.

### BACKGROUND

As reported in the 2017 Annual Data Report from the United States Renal Data System, there were over 700,000 reported cases of End Stage Renal Disease in 2015. Of these cases, over 87% began renal replacement therapy with hemodialysis. Renal replacement therapy with hemodialysis includes placing a dialysis catheter in a large vein such as the superior vena cava to allow for blood exchange between a patient and a hemodialysis machine. Since the placement of a dialysis catheter is generally for longer term use, complications can arise. Examples of such complications include thrombosis, embolisms, intracranial hemorrhages, major bleeding, mechanical catheter-related complications, and infections. Other examples can also include early catheter dysfunction, late or delayed dysfunction such as through the formation of a fibrin sheath on the outer surfaces of the catheter, and central vein stenosis. Additionally, guidewires are designed to navigate vessels to reach a lesion or vessel segment and guide larger catheters to the treatment site. One major complication with guidewires is the loss of guidewires. Each of these complications can results in staggering increases in cost and high mortality. To this extent, the current state of the art has failed to properly address these complications.

### SUMMARY

The disclosure generally relates to systems, apparatuses or devices, and methods for: conditioning surfaces of dialysis catheters; removing or clearing occlusions within dialysis catheters; or collecting tissue or cell samples.

In various embodiments are disclosed systems or devices for use with a catheter including: an elongated flexible guidewire capable of being slidably received within the catheter and including an end portion resiliently biased in a curved configuration, the end portion having a first section including a distal end of the guidewire and a second section adjacent to the first section, and an engaging member attached to a portion of the first section adjacent to the distal end.

In various embodiments are disclosed elongated flexible guidewires capable of being slidably received within the catheter and including an end portion resiliently biased in a curved configuration, the end portion having a first section including a distal end of the guidewire and a second section adjacent to the first section, and an engaging member attached to a portion of the first section adjacent to the distal end.

In various embodiments are disclosed kits for use with a catheter including: an elongated flexible guidewire including an end portion resiliently biased in a curved configuration, the end portion having a first section including a distal end of the guidewire and a second section adjacent to the first section, and an engaging member attached to a portion of the first section adjacent to the distal end; and a container with an interior containing the guidewire.

In various embodiments are disclosed methods of conditioning surfaces of a catheter including: providing an elongated flexible guidewire including an end portion resiliently biased in a curved configuration, the end portion having a first section including a distal end of the guidewire and a second section adjacent to the first section, and an engaging member attached to a portion of the first section adjacent to the distal end; inserting the guidewire into a proximal port of a catheter inserted into a blood vessel, where the proximal port is fluidly connected to a lumen defined by a wall and a distal port; and conditioning a surface of the wall by advancing the guidewire through the lumen, where the engaging member contacts the wall. In various embodiments, the methods of conditioning surfaces of a catheter can further include extending the engaging member past the distal port into the blood vessel, wherein the end portion biases towards the curved configuration.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a view of a flexible guidewire of various embodiments in a curved configuration.

FIG. 2 shows the end portion of a flexible guidewire of various embodiments transitioning between a curved configuration and straightened configuration.

FIG. 3 is a view showing the end portion of a guidewire of various embodiments in a curved configuration.

FIGS. 4, 5, 6, 7, 8, and 9 are views showing the end portion of a guidewire of various embodiments including bristles as an engaging member.

FIGS. 10 and 11 are views showing the end portion of a guidewire of various embodiments including a mesh substrate as an engaging member.

FIGS. 12 and 13 are views showing the end portion of a guidewire of various embodiments including fins as an engaging member.

FIGS. 14 and 15 are views showing the end portion of a guidewire of various embodiments including bristles and a mesh substrate as engaging members.

FIGS. 16 and 17 are views showing the end portion of a guidewire of various embodiments including bristles and fins as engaging members.

FIGS. 18 and 19 are views showing the end portion of a guidewire of various embodiments including fins and a mesh substrate as engaging members.

FIGS. 20 and 21 are views showing the end portion of a guidewire of various embodiments including bristles, fins, and mesh substrate as engaging members.

FIG. 22A is a view showing a system or device of various embodiments including a flexible guidewire, an end portion with an engaging member, and a torque device.

FIG. 22B is a view showing a system or device of various embodiments including a flexible guidewire, an end portion with an engaging member, a torque device, and a stop member positioned at a proximal end of the guidewire.

FIG. 23 is a view showing the stop member of a flexible guidewire of various embodiments position on the elongated member.

FIGS. 24 and 25 are views showing a stop member of various embodiments obstructing an opening of a catheter port.

FIGS. 26 and 27 are views showing a flexible guidewire of various embodiments being guided into a port of a dialysis catheter by a torque device that is capable of clamping on the guidewire. FIGS. 26 and 27 also show a stop member that is capable of obstructing further movement of the guidewire within the torque device.

FIGS. 28 and 29 are views showing a stop member of various embodiments capable of obstructing a portion of the flexible guidewire from entering the lumen of the torque device.

FIGS. 30, 31, 32, 33, and 34 are views showing kits of various embodiments.

FIG. 35 shows views of methods for using the systems/devices of various embodiments in which the end portion of the flexible guidewire is transitioned to the straightened configuration and inserted into an opening of a port of a catheter.

FIG. 36 shows a view of methods for using the systems/devices of various embodiments in which the engaging member contacts the inner surfaces of the shaft and dislodges debris such as cellular debris within the lumen of the catheter.

FIG. 37 shows views of methods for using the systems/devices of various embodiments in which the end portion of the guidewire or engaging member contacts an occlusion or thrombus and dislodges the occlusion or thrombus from the lumen and into a blood vessel.

FIG. 38 shows views of methods for using the systems/devices of various embodiments in which a user extends the engaging member past a distal port of the catheter and into the blood vessel, where the end portion biases towards the curved configuration.

FIG. 39 shows views of methods for using the systems/devices of various embodiments in which the flexible guidewire extending through the distal port of the catheter is moved to contact and remove debris/film attached to the outer surface of the catheter.

FIG. 40 shows a view of methods for using the systems/devices of various embodiments in which the flexible guidewire extending through the distal port of the catheter contacts and samples cells or tissues within a blood vessel.

FIG. 41 shows a view of methods for using the systems/devices of various embodiments in which an adapter is attached to the port of the catheter and the flexible guidewire is inserted through the adapter and into the opening of the port.

FIG. 42 shows views of methods for using the systems/devices of various embodiments in which the end portion of the guidewire or engaging member contacts an occlusion or thrombus and clears the occlusion or thrombus from the lumen of the catheter.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “or” can be understood to mean “at least one of”. The term “and” can also be understood to mean “at least one of” or “all”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The term “portion(s) of” can also be understood to include “some of”, “part of”, “all of”, or “the entirety of”. For example, “portion” can be understood to mean “at least a portion of”.

The terms “system(s)”, “device(s), “guidewire(s)”, or “flexible guidewire(s)” can be used interchangeably.

The term “occlusion” is understood to include any mass that blocks or impedes flow through a lumen of a catheter. For example, occlusions can include a thrombus, blood clot, an aggregation of platelets and red blood cells forming a plug, fluid, or precipitate that occlude, impede, or block flow through a lumen of a catheter.

The term “deposit(s)” is understood to be a build-up or formation of material on the outer surface of a catheter positioned within a blood vessel. The materials can include, for example, proteins, fluids, or precipitates. Examples of proteins include fibrin such as fibrin deposits or aggregates of red blood cells such as a thrombus (e.g. thrombotic deposits). Examples of fluids can include infusion fluids and examples of precipitates can include drug/pharmaceutical composition precipitates.

The disclosure generally relates to systems, apparatuses or devices, and methods for: conditioning surfaces of dialysis catheters; removing or clearing occlusions within dialysis catheters; or collecting tissue or cell samples.

In various embodiments are disclosed elongated flexible guidewires capable of being slidably received within the catheter and including an end portion resiliently biased in a curved configuration, the end portion having a first section including a distal end of the guidewire and a second section adjacent to the first section, and an engaging member attached to a portion of the first section adjacent to the distal end. Examples of guidewires such as J-tip guidewires are disclosed in U.S. Pat. Nos. 5,040,543; 5,437,288; 5,681,335; 5,851,189; 6,626,869; 8,308,658; 8,353,850; 8,696,695; and 9,968,761, each of which are incorporated in their entirety by reference.

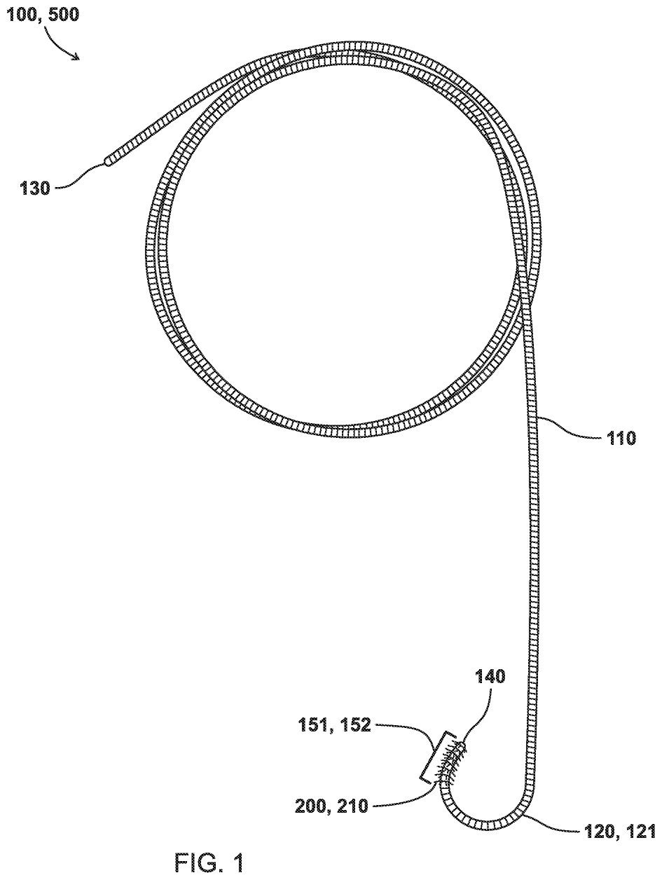

FIG. 1 is a view of a flexible guidewire 100 of various embodiments in a curved configuration. The flexible guidewire 100 includes an elongated member 110 and end portion 120 resiliently biased in a curved configuration 121. The elongated member 110 includes a proximal end 130 and the end portion 120 includes a distal end 140. As shown in FIG. 1, the elongated member 110 and end portion 120 biased in a curved configuration 121 has a shape similar to the letter “J” or can be a J-tip guidewire as understood in the art. FIG. 1 also shows a system or device 500 including the flexible guidewire 100.

The elongated member or end portion of various embodiments can have, for example, a core diameter/functional diameter 111 of 3 French Gauge (Fr), 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 23 Fr, 24 Fr, 25 Fr, 26 Fr, 27 Fr, 28 Fr, 29 Fr, 30 Fr, 31 Fr, 32 Fr, 33 Fr, 34 Fr, 0.5 millimeters (mm), 0.9 mm, 0.7 mm, 0.8 mm, 1 mm, 1.3 mm, 1.4 mm, 1.5 mm, 1.6 mm, 1.7 mm, 2.2 mm, 2.3 mm, 3.2 mm, 4 mm, 4.2 mm, 4.3 mm, 4.6 mm, 4.7 mm, 5 mm, 5.3 mm, 5.6 mm, 5.7 mm, 6 mm, 6.3 mm, 6.6 mm, 6.7 mm, 7.3 mm, 8 mm, 8.6 mm, 8.7 mm, 9.3 mm, 10 mm, 10.6 mm, 10.7 mm, 11.3 mm 0.014 inch (0.03556 centimeter (cm)), 0.018 inch (0.04572 cm), 0.025 inch (0.0635 cm), 0.032 inch (0.08128 cm), 0.035 inch (0.0889 cm), or 0.038 inch (0.09652 cm). In various embodiments, the core diameter/functional diameter is a range between any two diameters listed above.

The length of the elongated member of various embodiments is 80 cm, 100 cm, 120 cm, 130 cm, 140 cm, 150 cm, 160 cm, 170 cm, 180 cm, 190 cm, 200 cm, 210 cm, 220 cm, 230 cm, 240 cm, 250 cm, 260 cm, 270 cm, 280 cm, 290 cm, or 300 cm. In various embodiments, the length of the elongated member is a range between any two lengths listed above.

The length of the end portion of various embodiments is 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, or 10 cm. In various embodiments, the length of the end portion is a range between any two lengths listed above.

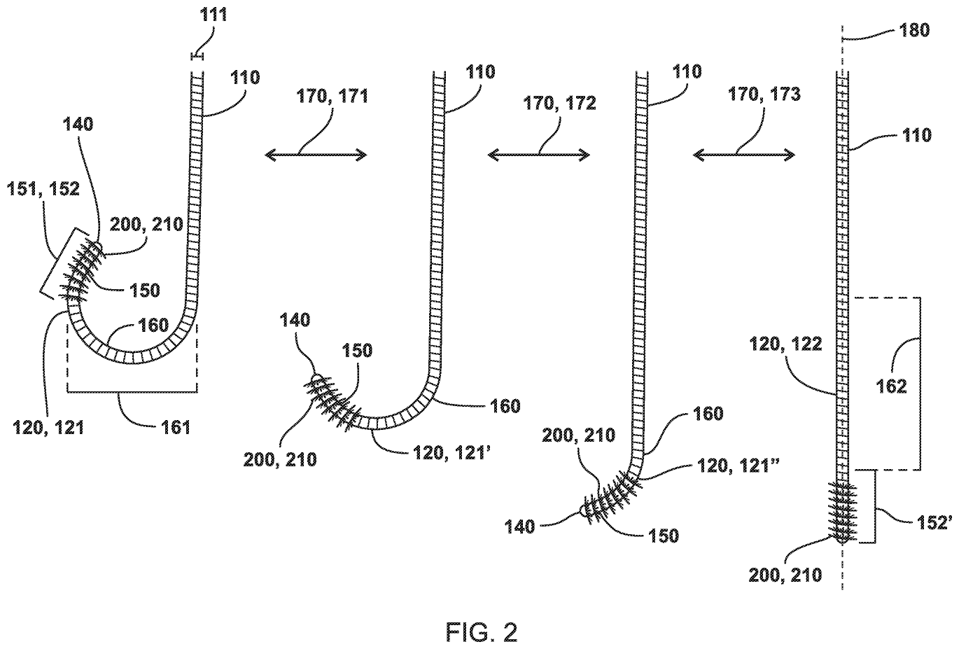

In various embodiments, the end portion is moveable between a curved configuration and a straightened configuration, where a user can engage the guidewire to move the end portion between the curved configuration and a straightened configuration. In various embodiments, the second section of the moveable member in the straightened configuration and a portion of the elongated member adjacent to the second section can share an axis. FIG. 2 shows the end portion of a flexible guidewire 100 of various embodiments transitioning 170,171,172,173 between a curved configuration 121,121′,121″ and straightened configuration 122. FIG. 2 also shows the core diameter/functional diameter 111 of the elongated member. The end portion 120 includes a first section 150 including a distal end 140 and a second section 160 positioned between the first section 150 and elongated member 110. The first section 150 or second section 160 can be generally curved 151,161 in the curved configuration 121. Alternatively, the first section 150 could be generally straight 152 in the curved configuration similar to when the end portion 120 is in the straightened configuration 122. In alternative embodiments, a portion of the first section is generally parallel to the elongated member.

As shown in FIG. 2, the first 150 and second 160 section of various embodiments are curved 151,161. In various embodiments, the radius of curvature for the curve 151 of the first section 150 is 1 millimeter (mm), 2 mm, 3 mm, 4 mm, 5 mm, 6 mm, 7 mm, 8 mm, 9 mm, 10 mm, 11 mm, 12 mm, 13 mm, 14 mm, or 15 mm. In various embodiments, the radius of curvature for the curve 161 of the second section 160 is 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm. In various embodiments, the radius of curvature for the first or second section is a range between any two measurements listed above. In various embodiments, a portion of the first section has a radius of curvature that is greater than a radius of curvature of the second section in the curved configuration.

In straightened configuration 122, a portion of the first 150 or second 160 section of various embodiments can share an axis 180 with the elongated member.

In various embodiments, the length of the straightened first section 150,152,152′ is 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, or 9.5 cm. In various embodiments, the length of the straightened second section 160, 162 is 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, or 9.5 cm. In various embodiments, the first section has a length less than a length of the second section in the straightened configuration.

In various embodiments, the guidewire 100 includes an engaging member 200 attached to a portion of the first section 150 adjacent to the distal end 140. In various embodiments, the engaging member is not attached to the second section. The engaging member 200 of various embodiments extends from the first section 150 and is capable of contacting surfaces of a catheter 300 including shaft(s) 305 with inner surfaces 310 defining a lumen 320 and outer surfaces 340. In various embodiments, the engaging member 200 is adaptable to allow the guidewire 100 to be positioned within the lumen 320 or to allow the guidewire 100 to move within the lumen 320. For example, the engaging member 200 is adaptable to allow the guidewire 100 to move along an axis 321 within the lumen 320 when the lumen 320 is in a generally straightened configuration 322.

As shown in FIGS. 1-23, examples of the engaging member 200 can include bristle(s) 210, mesh substrate(s) 220, fin(s) 230, or combinations thereof.

In various embodiments, the bristles are attached to the portion of the first section at angles of 90 degrees (i.e. 90°) or more relative to an outer surface of the portion of the first section and the distal end in the curved configuration. In various embodiments, the bristles are attached to the portion of the first section at angles of 90°, 91°, 92°, 93°, 94°, 95°, 96°, 97°, 98°, 99°, 100°, 101°, 102°, 103°, 104°, 105°, 106°, 107°, 108°, 109°, 110°, 111°, 112°, 113°, 114°, 115°, 116°, 117°, 118°, 119°, 120°, 121°, 122°, 123°, 124°, 125°, 126°, 127°, 128°, 129°, 130°, 131°, 132°, 133°, 134°, 135°, 136°, 137°, 138°, 139°, 140°, 141°, 142°, 143°, 144°, 145°, 146°, 147°, 148°, 149°, 150°, 151°, 152°, 153°, 154°, 155°, 156°, 157°, 158°, 159°, 160°, 161°, 162°, 163°, 164°, 165°, 166°, 167°, 168°, 169°, 170°, 171°, 172°, 173°, 174°, 175°, 176°, 177°, 178°, 179°, or 180° relative to an outer surface of the portion of the first section and the distal end in the curved configuration. In various embodiments, the angles are a range between any two angles listed above. Examples of bristle attachments are disclosed in U.S. Pat. Nos. 5,681,335; 6,725,492; and 6,920,662, each of which are incorporated in their entirety by reference.

The bristles of various embodiments have a strength sufficient for allowing the guidewire to dislodge materials deposited on a surface of a catheter or blocking a lumen of the catheter or to extract cells and tissues from, for example, a wall of a blood vessel or blood within the blood vessel. The bristles of various embodiments are also flexible/adaptable to allow for the guidewire to be inserted into and moved through a lumen of a catheter. Further, the flexibility/adaptability of the bristles of various embodiments does not damage the catheter when the guidewire is inserted or through the lumen of the catheter. The bristles of various embodiments may include natural materials, synthetic materials, metals, plastics, or carbon-based materials. The materials of various embodiments are materials acceptable for use in organisms such as human being or mammals or are biocompatible materials. Examples of such materials can include polyolefins such as polypropylenes and polyethylenes; polyamides such as nylons; polyesters such as polyethylene terephthalate; polymethylmethacrylate; styrene acrylonitrate; cellulose esters such as cellulose propionate; block copolymers include styrenes (for example styrene ethylene butadiene styrene, or styrene butadiene styrene), polyolefins (for example polypropylene/ethylene propylene diamine modified systems (i.e. synthetic rubber)), polyamides (for example polyamide (2 or polyamide 6), polyesters (for example polyester ester or polyether ester), polyurethanes (for example polyesterurethane, polyetherurethane or polyesteretherurethane); polyketones (e.g., polyether ether ketones (PEEK), polyaryl ether ketones (PAEK) or mixtures thereof), polyphenylene sulfide (PPS); natural plant-based fibers; or metals such as titanium, aluminum, brass, steel, carbon steel, or stainless steel.

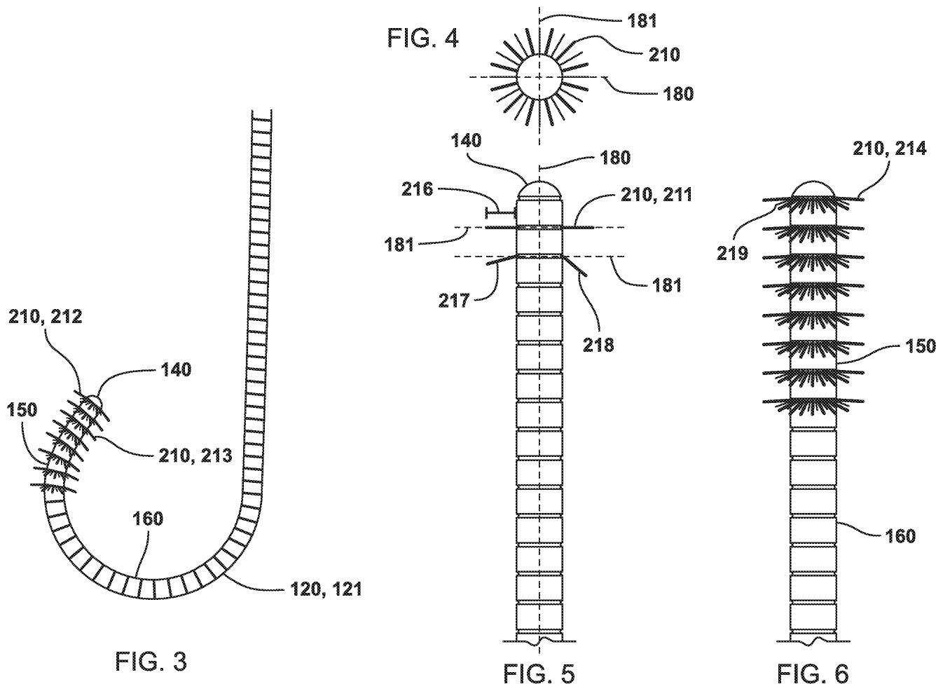

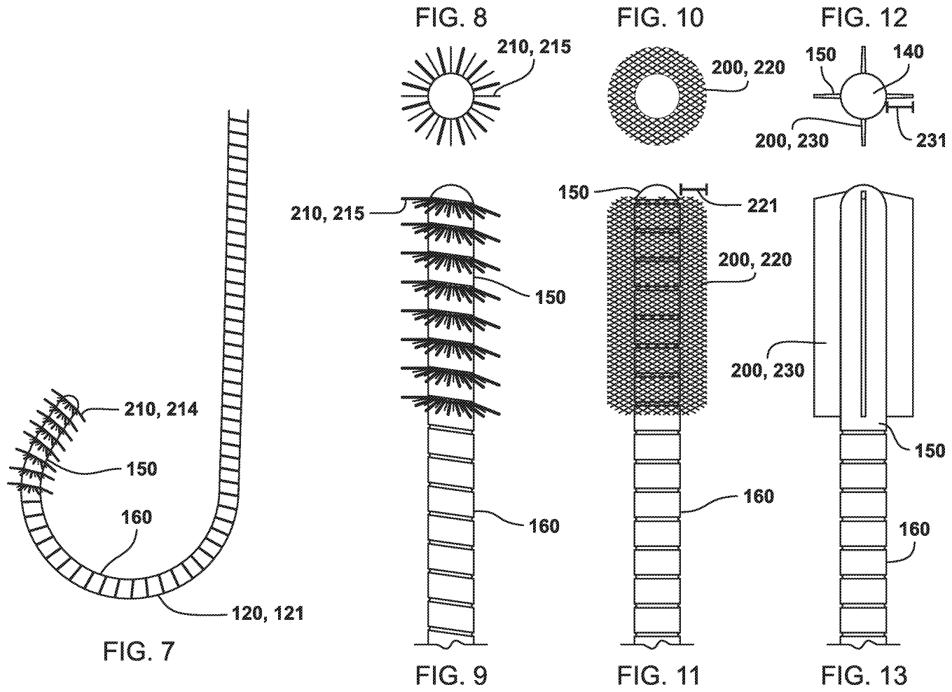

In various embodiments, the engaging member is arranged in a partially annular configuration or an annular configuration around the first section. FIGS. 3, 4, and 5 show the first section 150 of an end portion 120 of various embodiments in a curved configuration with bristles 210 extending at different angles 211,212,213,217,218,219. For example, bristle 211 can extend along axis 181 and perpendicularly to axis 180, whereas other bristles 212,213,217,218,219 can extend away from the distal end 140. Also as shown in FIG. 5, the bristles 210 can be arranged annularly around the first section 150. FIGS. 6 and 7 further shows examples of a grouping or tuft of bristles 214 of bristles 210 arranged around the first section 150.

FIG. 5 further shows the length 216 of the bristles 210. In various embodiments, the length of the bristles 0.25 mm, 0.5 mm, 0.75 mm, 1 mm, 1.25 mm, 1.5 mm, 1.75 mm, 2 mm, 2.25 mm, 2.5 mm, 2.75 mm, 3 mm, 3.25 mm, 3.5 mm, 3.75 mm, 4 mm, 4.25 mm, 4.5 mm, 4.75 mm, 5 mm, 5.25 mm, 5.5 mm, 5.75 mm, 6 mm, 6.25 mm, 6.5 mm, 6.75 mm, 7 mm, 7.25 mm, 7.5 mm, 7.75 mm, 8 mm, 8.25 mm, 8.5 mm, 8.75 mm, 9 mm, 9.25 mm, 9.5 mm, 9.75 mm, 10 mm, 10.25 mm, 10.5 mm, 10.75 mm, 11 mm, 11.25 mm, 11.5 mm, 11.75 mm, 12 mm, 12.25 mm, 12.5 mm, 12.75 mm, 13 mm, 13.25 mm, 13.5 mm, 13.75 mm, 14 mm, 14.25 mm, 14.5 mm, 14.75 mm, 15 mm, 15.25 mm, 15.5 mm, 15.75 mm, 16 mm, 16.25 mm, 16.5 mm, 16.75 mm, 17 mm, 17.25 mm, 17.5 mm, 17.75 mm, 18 mm, 18.25 mm, 18.5 mm, 18.75 mm, 19 mm, 19.25 mm, 19.5 mm, 19.75 mm, or 20 mm. In various embodiments, the length of the bristles is a range between any two lengths listed above.

The bristles of various embodiments can be arranged in various arrangements. For example as shown in FIGS. 8 and 9, grouping or tufts 215 of bristles 210 are arranged in a spiraling pattern.

In various embodiments, the engaging member includes a mesh substrate attached to the portion of the first section. The mesh substrate of various embodiments has a strength sufficient for allowing the guidewire to dislodge materials deposited on a surface of a catheter or blocking a lumen of the catheter or to extract cells and tissues from, for example, a wall of a blood vessel or blood within the blood vessel. The mesh substrate of various embodiments is also flexible/adaptable to allow for the guidewire to be inserted into and moved through a lumen of a catheter. Further, the flexibility/adaptability of the mesh substrate of various embodiments does not damage the catheter when the guidewire is inserted or moved through the lumen of the catheter. For example, the mesh substrate can be rigid, semi-rigid, or soft. The mesh substrate of various embodiments may include natural materials, synthetic materials, metals, plastics, or carbon-based materials. The materials of various embodiments are materials acceptable for use in organisms such as human being or mammals or are biocompatible materials. Examples of such materials can include polyolefins such as polypropylenes and polyethylenes; polyamides such as nylons; polyesters such as polyethylene terephthalate; polymethylmethacrylate; styrene acrylonitrate; cellulose esters such as cellulose propionate; block copolymers include styrenes (for example styrene ethylene butadiene styrene, or styrene butadiene styrene), polyolefins (for example polypropylene/ethylene propylene diamine modified systems (i.e. synthetic rubber)), polyamides (for example polyamide (2 or polyamide 6), polyesters (for example polyester ester or polyether ester), polyurethanes (for example polyesterurethane, polyetherurethane or polyesteretherurethane); polyketones (e.g., polyether ether ketones (PEEK), polyaryl ether ketones (PAEK) or mixtures thereof), polyphenylene sulfide (PPS); natural plant-based fibers; or metals such as titanium, aluminum, brass, steel, carbon steel, or stainless steel.

FIGS. 10 and 11 show engaging member 200 including a mesh substrate 220. The mesh substrate 220 can, for example, extend from the first section 150 in an annular configuration around a portion of the first section 150. FIG. 11 further shows a length 221 of the mesh substrate 220 extending from the first section 150. In various embodiments, the length of the mesh substrate extending from the first section is 0.25 mm, 0.5 mm, 0.75 mm, 1 mm, 1.25 mm, 1.5 mm, 1.75 mm, 2 mm, 2.25 mm, 2.5 mm, 2.75 mm, 3 mm, 3.25 mm, 3.5 mm, 3.75 mm, 4 mm, 4.25 mm, 4.5 mm, 4.75 mm, 5 mm, 5.25 mm, 5.5 mm, 5.75 mm, 6 mm, 6.25 mm, 6.5 mm, 6.75 mm, 7 mm, 7.25 mm, 7.5 mm, 7.75 mm, 8 mm, 8.25 mm, 8.5 mm, 8.75 mm, 9 mm, 9.25 mm, 9.5 mm, 9.75 mm, 10 mm, 10.25 mm, 10.5 mm, 10.75 mm, 11 mm, 11.25 mm, 11.5 mm, 11.75 mm, 12 mm, 12.25 mm, 12.5 mm, 12.75 mm, 13 mm, 13.25 mm, 13.5 mm, 13.75 mm, 14 mm, 14.25 mm, 14.5 mm, 14.75 mm, 15 mm, 15.25 mm, 15.5 mm, 15.75 mm, 16 mm, 16.25 mm, 16.5 mm, 16.75 mm, 17 mm, 17.25 mm, 17.5 mm, 17.75 mm, 18 mm, 18.25 mm, 18.5 mm, 18.75 mm, 19 mm, 19.25 mm, 19.5 mm, 19.75 mm, or 20 mm. In various embodiments, the length 221 of the mesh substrate extending from the first section is a range between any two lengths listed above.

In various embodiments, the engaging member includes a fin attached to the portion of the first section. The fin of various embodiments has a strength sufficient for allowing the guidewire to dislodge materials deposited on a surface of a catheter or blocking a lumen of the catheter or to extract cells and tissues from, for example, a wall of a blood vessel or blood within the blood vessel. The fin of various embodiments is also flexible/adaptable to allow for the guidewire to be inserted into and moved through a lumen of a catheter. Further, the flexibility/adaptability of the fin of various embodiments does not damage the catheter when the guidewire is inserted or moved through the lumen of the catheter. The fin of various embodiments may include natural materials, synthetic materials, metals, plastics, or carbon-based materials. The materials of various embodiments are materials acceptable for use in organisms such as human being or mammals or are biocompatible materials. Examples of such materials can include polyolefins such as polypropylenes and polyethylenes; polyamides such as nylons; polyesters such as polyethylene terephthalate; polymethylmethacrylate; styrene acrylonitrate; cellulose esters such as cellulose propionate; block copolymers include styrenes (for example styrene ethylene butadiene styrene, or styrene butadiene styrene), polyolefins (for example polypropylene/ethylene propylene diamine modified systems (i.e. synthetic rubber)), polyamides (for example polyamide (2 or polyamide 6), polyesters (for example polyester ester or polyether ester), polyurethanes (for example polyesterurethane, polyetherurethane or polyesteretherurethane); polyketones (e.g., polyether ether ketones (PEEK), polyaryl ether ketones (PAEK) or mixtures thereof), polyphenylene sulfide (PPS); natural plant-based fibers; or metals such as titanium, aluminum, brass, steel, carbon steel, or stainless steel.

FIGS. 12 and 13 show engaging member 200 including fins 230. FIG. 12 further shows a length 231 of the fin 230 extending from the first section 150. In various embodiments, the length of the fin extending from the first section is 0.25 mm, 0.5 mm, 0.75 mm, 1 mm, 1.25 mm, 1.5 mm, 1.75 mm, 2 mm, 2.25 mm, 2.5 mm, 2.75 mm, 3 mm, 3.25 mm, 3.5 mm, 3.75 mm, 4 mm, 4.25 mm, 4.5 mm, 4.75 mm, 5 mm, 5.25 mm, 5.5 mm, 5.75 mm, 6 mm, 6.25 mm, 6.5 mm, 6.75 mm, 7 mm, 7.25 mm, 7.5 mm, 7.75 mm, 8 mm, 8.25 mm, 8.5 mm, 8.75 mm, 9 mm, 9.25 mm, 9.5 mm, 9.75 mm, 10 mm, 10.25 mm, 10.5 mm, 10.75 mm, 11 mm, 11.25 mm, 11.5 mm, 11.75 mm, 12 mm, 12.25 mm, 12.5 mm, 12.75 mm, 13 mm, 13.25 mm, 13.5 mm, 13.75 mm, 14 mm, 14.25 mm, 14.5 mm, 14.75 mm, 15 mm, 15.25 mm, 15.5 mm, 15.75 mm, 16 mm, 16.25 mm, 16.5 mm, 16.75 mm, 17 mm, 17.25 mm, 17.5 mm, 17.75 mm, 18 mm, 18.25 mm, 18.5 mm, 18.75 mm, 19 mm, 19.25 mm, 19.5 mm, 19.75 mm, or 20 mm. In various embodiments, the length 231 of the fin extending from the first section is a range between any two lengths listed above.

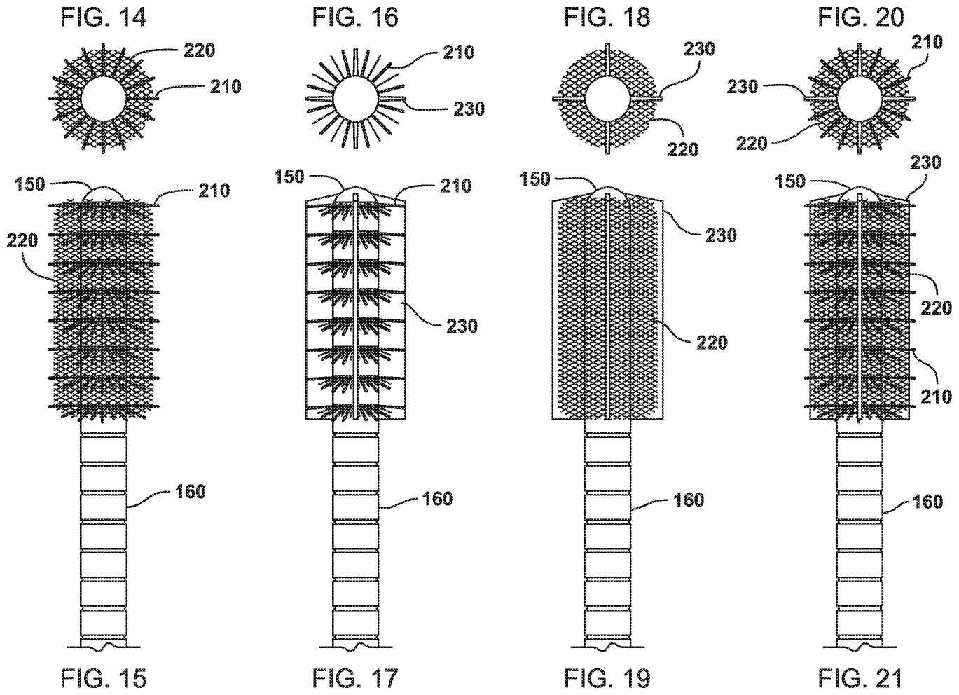

In various embodiments, the engaging member includes a plurality of different members such as bristle(s), mesh substrate(s), or fin(s). FIGS. 14-21 shows various embodiments of the guidewires including combinations of different engaging members FIGS. 15 and 16 show bristles 210 and mesh substrate 220 extending from the first section 150. FIGS. 17 and 18 show bristles 210 and fins 230 extending from the first section 150. FIGS. 19 and 20 show fins 230 and a mesh substrate 220 extending from the first section 150. FIGS. 20 and 21 show bristles 210, mesh substrate 220, and fins 230 extending from the first section 150.

In various embodiments are disclosed systems or devices for use with a catheter including: an elongated flexible guidewire capable of being slidably received within the catheter and including an end portion resiliently biased in a curved configuration, the end portion having a first section including a distal end of the guidewire and a second section adjacent to the first section, and an engaging member attached to a portion of the first section adjacent to the distal end. The guidewire of various embodiments can further include a plurality of evenly spaced indicia visible to identify measured distances from the distal end.

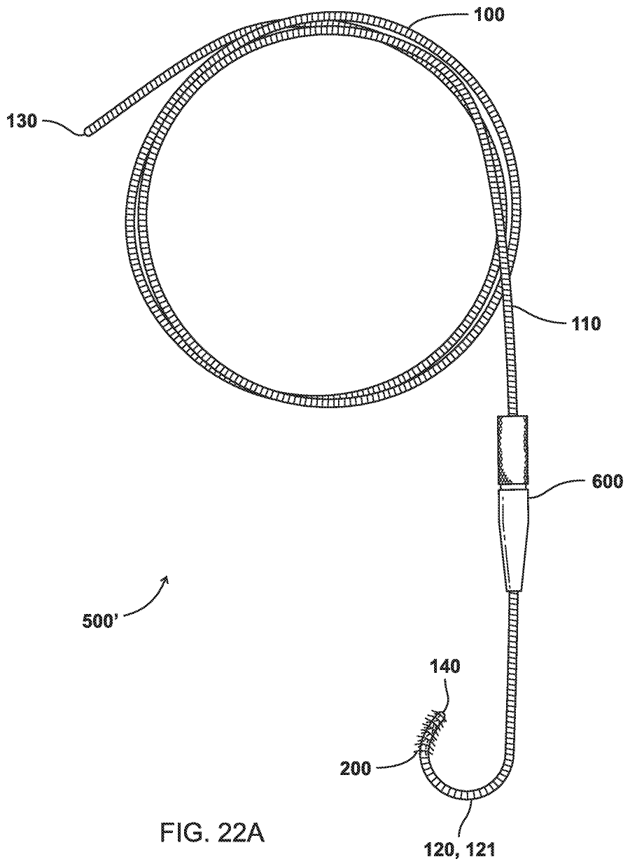

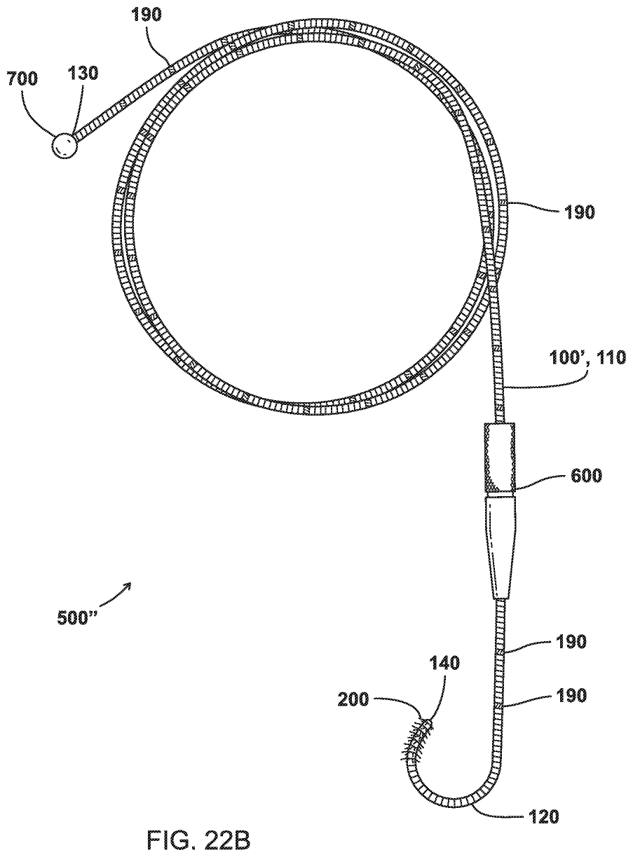

FIG. 22A is a view showing a system or device 500′ of various embodiments including a flexible guidewire 100, an end portion 120 with an engaging member 200 in a curved configuration 121, and a torque device 600.

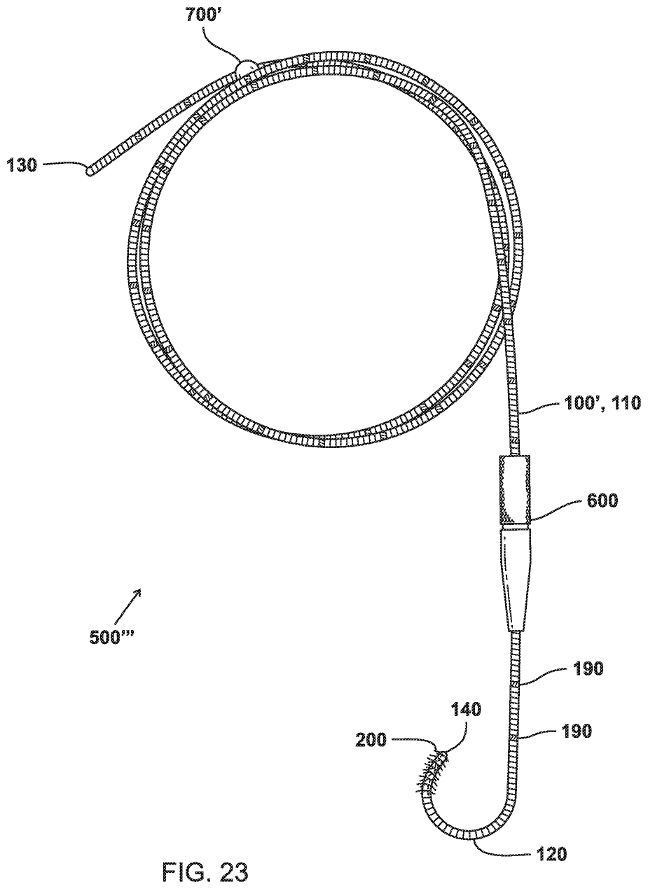

FIGS. 22B and 23 are views showing systems or devices 500″,500″′ of various embodiments including a flexible guidewire 100′, an end portion 120 with an engaging member 200, a torque device 600, and a stop member 700,700′ positioned at or near a proximal end 130 of the elongated member 110. The flexible guidewire 100′ has a plurality of evenly spaced indicia visible 190 to identify measured distances from the distal end. In various embodiments, the evenly spaced indicia is spaced apart at intervals of 0.1 cm, 0.25 cm, 0.5 cm, 0.75 cm, 1 cm, 1.25 cm, 1.5 cm, 1.75 cm, 2 cm, 2.25 cm, 2.5 cm, 2.75 cm, 3 cm, 3.25 cm, 3.5 cm, 3.75 cm, 4 cm, 4.25 cm, 4.5 cm, 4.75 cm, 5 cm, 5.25 cm, 5.5 cm, 5.75 cm, 6 cm, 6.25 cm, 6.5 cm, 6.75 cm, 7 cm, 7.25 cm, 7.5 cm, 7.75 cm, 8 cm, 8.25 cm, 8.5 cm, 8.75 cm, 9 cm, 9.25 cm, 9.5 cm, 9.75 cm, or 10 cm. The intervals of various embodiments are a range between any two intervals listed above.

Examples of indicia can include etchings or coatings on the guidewire that are visible to a user. In other examples, the indicia can include color(s) or measurement marking(s) such a “0.5” for 0.5 cm from the distal end or “1” for 1 cm from the distal end. Further examples of indicia are also disclosed in U.S. Pat. Nos. 5,084,022; 6,613,002; U.S. Patent Application Publication No. 2005/0148902; and PCT Application Publication No. WO2002/047549, each of which are incorporated in their entirety by reference.

In various embodiments, the system or device further includes a stop member attached to the guidewire and configured to obstruct a portion or the distal end of the guidewire from entering the catheter. FIG. 22B shows the stop member 700 attached to the proximal end 130 of the elongated member 110. FIG. 23 shows the proximal stop member 700′ attached to the elongated member 110 at a position near or adjacent to the proximal end 130.

In various embodiments, the stop member 700,700′ has a diameter 701 or shape to obstruct the stop member or the elongated member from entering a port 330 or lumen 320 of a catheter. The types of catheters can include catheters with lumen diameters of 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 23 Fr, 24 Fr, 25 Fr, 26 Fr, 27 Fr, 28 Fr, 29 Fr, 30 Fr, 31 Fr, 32 Fr, 33 Fr, 34 Fr, 0.5 mm, 0.9 mm, 0.7 mm, 0.8 mm, 1 mm, 1.3 mm, 1.4 mm, 1.5 mm, 1.6 mm, 1.7 mm, 2.2 mm, 2.3 mm, 3.2 mm, 4 mm, 4.2 mm, 4.3 mm, 4.6 mm, 4.7 mm, 5 mm, 5.3 mm, 5.6 mm, 5.7 mm, 6 mm, 6.3 mm, 6.6 mm, 6.7 mm, 7.3 mm, 8 mm, 8.6 mm, 8.7 mm, 9.3 mm, 10 mm, 10.6 mm, 10.7 mm, 11.3 mm 0.014 inch (0.03556 cm), 0.018 inch (0.04572 cm), 0.025 inch (0.0635 cm), 0.032 inch (0.08128 cm), 0.035 inch (0.0889 cm), or 0.038 inch (0.09652). In various embodiments, the lumen diameter is a range between any two diameters listed above. Alternatively, the diameter 701 of various embodiments is or is greater than 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 23 Fr, 24 Fr, 25 Fr, 26 Fr, 27 Fr, 28 Fr, 29 Fr, 30 Fr, 31 Fr, 32 Fr, 33 Fr, 34 Fr, 0.5 mm, 0.9 mm, 0.7 mm, 0.8 mm, 1 mm, 1.3 mm, 1.4 mm, 1.5 mm, 1.6 mm, 1.7 mm, 2.2 mm, 2.3 mm, 3.2 mm, 4 mm, 4.2 mm, 4.3 mm, 4.6 mm, 4.7 mm, 5 mm, 5.3 mm, 5.6 mm, 5.7 mm, 6 mm, 6.3 mm, 6.6 mm, 6.7 mm, 7.3 mm, 8 mm, 8.6 mm, 8.7 mm, 9.3 mm, 10 mm, 10.6 mm, 10.7 mm, 11.3 mm 0.014 inch (0.03556 cm), 0.018 inch (0.04572 cm), 0.025 inch (0.0635 cm), 0.032 inch (0.08128 cm), 0.035 inch (0.0889 cm), or 0.038 inch (0.09652 cm). In various embodiments, the diameter 701 is a range between any two diameters listed above.

The stop member 700,700′ of various embodiments may include natural materials, synthetic materials, metals, plastics, or carbon-based materials. The materials of various embodiments are materials acceptable for use in organisms such as human being or mammals or are biocompatible materials. Examples of such materials can include polyolefins such as polypropylenes and polyethylenes; polyamides such as nylons; polyesters such as polyethylene terephthalate; polymethylmethacrylate; styrene acrylonitrate; cellulose esters such as cellulose propionate; block copolymers include styrenes (for example styrene ethylene butadiene styrene, or styrene butadiene styrene), polyolefins (for example polypropylene/ethylene propylene diamine modified systems (i.e. synthetic rubber)), polyamides (for example polyamide (2 or polyamide 6), polyesters (for example polyester ester or polyether ester), polyurethanes (for example polyesterurethane, polyetherurethane or polyesteretherurethane); polyketones (e.g., polyether ether ketones (PEEK), polyaryl ether ketones (PAEK) or mixtures thereof), polyphenylene sulfide (PPS); natural plant-based fibers; or metals such as titanium, aluminum, brass, steel, carbon steel, or stainless steel.

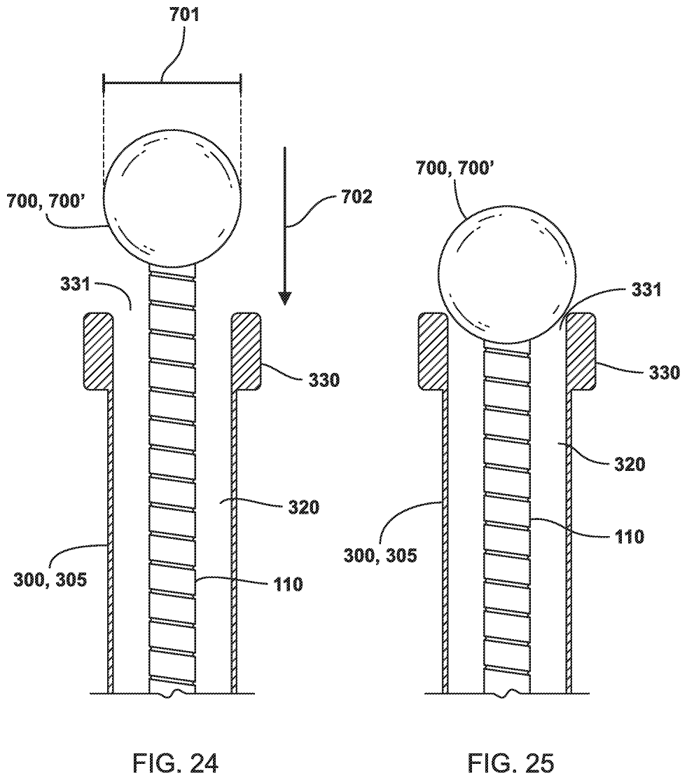

FIGS. 24 and 25 are views showing the stop member 700,700′ of various embodiments obstructing an opening 331 of a port 330 of a catheter 300 As shown in FIG. 24, the elongated member 110 is inserted into the opening 331 of the port 330 and moved 702 through the lumen 320 of the catheter. As shown in FIG. 25, the stop member 700,700′ can obstruct a distal end or a portion of the elongated member 110 from entering the opening 331 or the lumen 320.

In various embodiments, the system or device further includes a torque device including an elongated body having a lumen capable of receiving the guidewire and an end portion sized or shaped for insertion into a port of the catheter; an actuator operably connected to the elongated body and having a lumen configured for selective engagement of the guidewire, where the actuator is movable between a position where the guidewire is secured relative to the torque device and a position where the guidewire can movable relative to the torque device. The system of various embodiments can also include a stop member capable of obstructing further movement of the guidewire within the torque device.

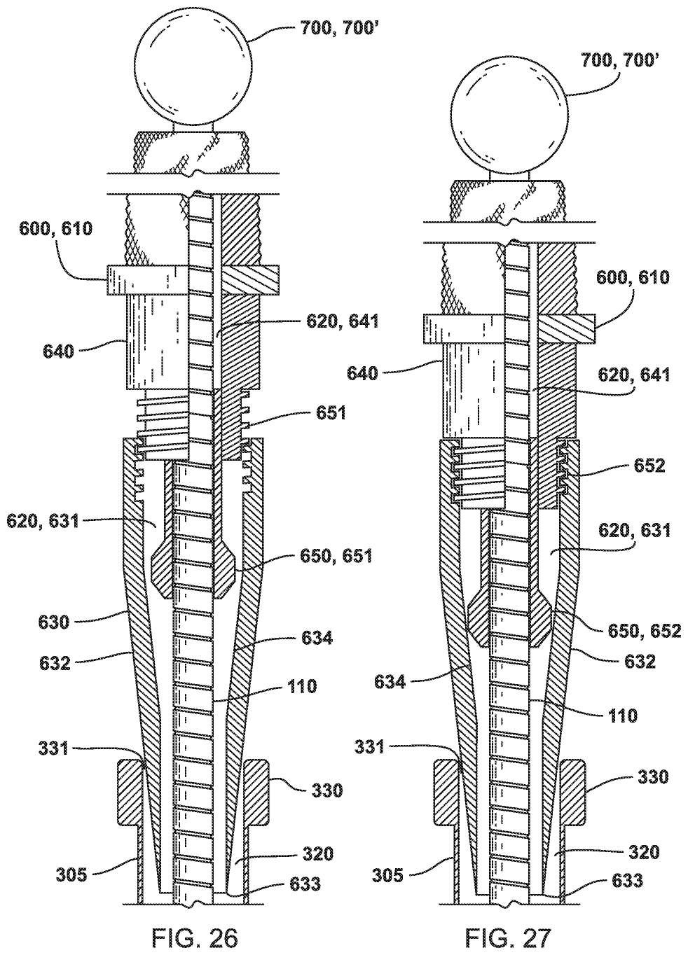

FIGS. 26 and 27 are views showing a flexible guidewire 100,100′ of various embodiments being guided into an opening 331 of a port 330 of a dialysis catheter 300 via a torque device 600 that is capable of clamping on the flexible guidewire 100,100′. As shown in FIGS. 26 and 27, the torque device 600 has an elongated body 610 with a lumen 620 capable of receiving the flexible guidewire 100,100′. The elongated body 610 includes an end portion/end member 630 threadably coupled to a base member 640, where the end member 630 and base member 640 have bores 631,641 defining the lumen 620. The terms “end portion”, “end member”, “adapter”, “adapting member”, or “adapting portion” are used interchangeably.

The end portion of various embodiments is sized or shaped such that a portion of the end portion is capable of being inserted into an opening of a port of a catheter. In various embodiments, a percentage of the end portion that can be inserted past the opening of a port of a catheter is 10%, 15%, 20%, 25%, 30%, 35%, 40%, 45%, 50%, 55%, 60%, 65%, 70%, 75%, 80%, 85%, 90%, 95%, or 100% of the end portion. In various embodiments, the percentage of the end portion than can be inserted past the opening of a port of a catheter is a range between any two percentages listed above.

As shown in FIGS. 26 and 27, the end member 630 is inserted through the opening 331 of the port 330 and allows for the guidewire 100,100′ to be moved through the bore 631, past the opening 331, and into the lumen 320 of the catheter 300. The end member 630 as shown in FIGS. 26 and 27 has a conical shape with a slanting outer surface 632. The diameter of the end member 630 gradually increases, where the diameter of the end member 630 is the smallest at a distal end 633 that allows for easier insertion into the opening 331. The end member 630 is inserted past the opening to a point where the outer surface 632 contacts the port 330 or the shaft 305 such that the end member 630 is capable of being supported by the catheter 300. To this end, the end member serves as or is an adapter, adapting member, or adapting portion for inserting the flexible guidewire into the catheter since having the end member inserted into the catheter prior to the insertion of the flexible guidewire can allow for greater ease in inserting the flexible guidewire into the catheter.

As previously stated, the end member 630 is threadably coupled to the base member 640. The torque device 600 further includes a collet 650 connect to the base member 640 and positioned within bore 631 of the end member 630. Through the threadable connection, the end member 630 and base member 640 are movable between a first position 651, where the collet 650 is positioned away from the guidewire 100,100′ such that the guidewire 100,100′ can be move within the lumen 620 relative to the torque device 600, and a second position 652, where the end member 630 biases the collet 650 to contact the guidewire 100,100′ such that the guidewire 100,100′ is clamped to torque device 600 and the guidewire 100,100′ does be move within the lumen 620 relative to the torque device 600. For example, the end member 630 or base member 640 is rotatable relative to the other such that rotation of the end member 630 or base member 640 relative to the other transitions the torque device 600 between the first 651 and second 652 positions. FIG. 26 shows the end member 630 and base member 640 in the first position 651. The bore 631 of the end member 630 has a conical shape defined by a slanting inner surface 634. The diameter of the bore 631 gradually increases, where the diameter of the bore 631 is the smallest at a distal end 633. FIG. 27 shows the end member 630 and base member 640 in the second position 652. The position of the end member 630 in the second position 652 is closer to the base member 640 relative to a position of the end member 630 to the base member 640 in the first position 651. In the second position 652, the inner surface 634 of the end member 630 contacts and biases the collet 650 against the guidewire 100,100′. Also, the stop member 700,700′ as shown in FIGS. 26 and 27 has a diameter 701 or shape to obstruct the stop member 700,700′ or the elongated member 110 from entering the bore 641 of the base member 640.

In various embodiments, the bores of the end portion or base member has a diameter of or greater than 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 23 Fr, 24 Fr, 25 Fr, 26 Fr, 27 Fr, 28 Fr, 29 Fr, 30 Fr, 31 Fr, 32 Fr, 33 Fr, 34 Fr, 0.5 mm, 0.9 mm, 0.7 mm, 0.8 mm, 1 mm, 1.3 mm, 1.4 mm, 1.5 mm, 1.6 mm, 1.7 mm, 2.2 mm, 2.3 mm, 3.2 mm, 4 mm, 4.2 mm, 4.3 mm, 4.6 mm, 4.7 mm, 5 mm, 5.3 mm, 5.6 mm, 5.7 mm, 6 mm, 6.3 mm, 6.6 mm, 6.7 mm, 7.3 mm, 8 mm, 8.6 mm, 8.7 mm, 9.3 mm, 10 mm, 10.6 mm, 10.7 mm, 11.3 mm 0.014 inch (0.03556 cm), 0.018 inch (0.04572 cm), 0.025 inch (0.0635 cm), 0.032 inch (0.08128 cm), 0.035 inch (0.0889 cm), or 0.038 inch (0.09652 cm). In various embodiments, the diameter of the bores is a range between any two diameters listed above.

The torque device of various embodiments allows a user to manipulate the flexible guidewire including the position of the flexible guidewire within a lumen of a catheter or a position of the end portion relative to the catheter or within a blood vessel. Alternatively, the torque device of various embodiments can include torque devices disclosed in U.S. Pat. Nos. 7,831,297 and 7,186,224, each of which are incorporated in their entirety by reference.

In various embodiments, the system or device further includes a stop member attached to the guidewire and configured to obstruct a portion of the guidewire from entering the lumen of the elongated body of the torque device. In various embodiments, the portion of the guidewire can include the end portion of the guidewire or the distal end of the guidewire.

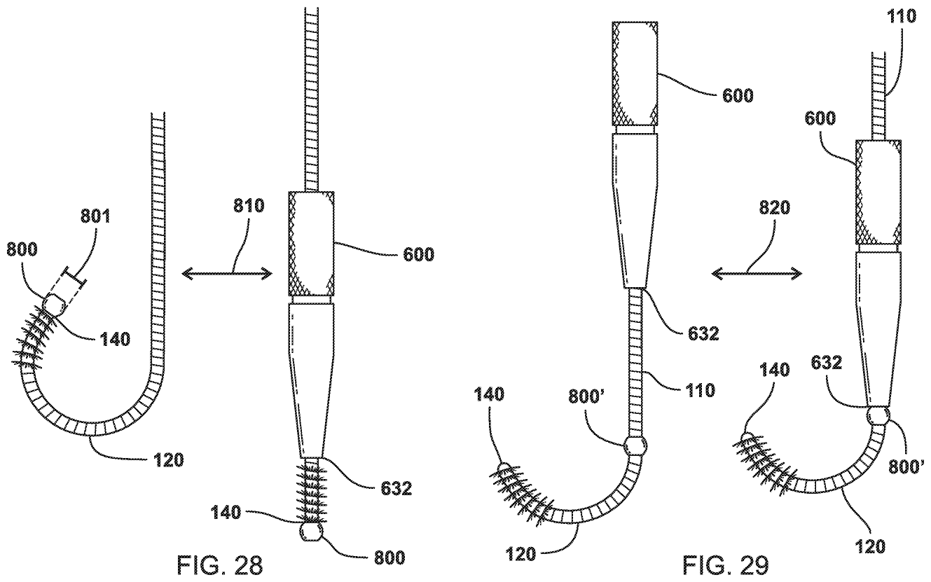

FIGS. 28 and 29 are views showing a stop member 800,800′ of various embodiments obstructing capable of preventing a portion of the flexible guidewire such as the end portion 120 from entering the lumen 620 of the torque device 600.

FIG. 28 shows the stop member 800 positioned on the distal end 140 of the guidewire 100,100′. When the torque device 600 is coupled to guidewire 100,100′ and is slidably moved 810 to the distal end 140, the stop member 800 obstructs the distal end 140 from entering the lumen 620 of the torque device 600. The obstruction can occur, for example, when the stop member 800 contacts the end 633 of the end member 630.

FIG. 29 shows the stop member 800′ positioned on the guidewire 100,100′, for example, adjacent to the end portion 120 of the guidewire 100,100′. When the torque device 600 is coupled to guidewire 100,100′ and is slidably moved 820 to the distal end 140, the stop member 800′ obstructs the distal end 140 from entering the lumen 620 of the torque device 600. The obstruction can occur, for example, when the stop member 800′ contacts the end 633 of the end member 630.

In various embodiments, the stop member 800,800′ has a diameter 801 or shape to obstruct a portion of the guidewire 100,100′ such as the distal end 140 or end portion 120 from entering the lumen 620 of the torque device 600. In various embodiments, the diameter 801 is greater than a diameter of the bore 631 at the distal end 633. In other embodiments, the diameter 801 of the stop member 800,800′ is, is greater than, or is less than 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 23 Fr, 24 Fr, 25 Fr, 26 Fr, 27 Fr, 28 Fr, 29 Fr, 30 Fr, 31 Fr, 32 Fr, 33 Fr, 34 Fr, 0.5 mm, 0.9 mm, 0.7 mm, 0.8 mm, 1 mm, 1.3 mm, 1.4 mm, 1.5 mm, 1.6 mm, 1.7 mm, 2.2 mm, 2.3 mm, 3.2 mm, 4 mm, 4.2 mm, 4.3 mm, 4.6 mm, 4.7 mm, 5 mm, 5.3 mm, 5.6 mm, 5.7 mm, 6 mm, 6.3 mm, 6.6 mm, 6.7 mm, 7.3 mm, 8 mm, 8.6 mm, 8.7 mm, 9.3 mm, 10 mm, 10.6 mm, 10.7 mm, 11.3 mm 0.014 inch (0.03556 cm), 0.018 inch (0.04572 cm), 0.025 inch (0.0635 cm), 0.032 inch (0.08128 cm), 0.035 inch (0.0889 cm), or 0.038 inch (0.09652 cm). In various embodiments, the diameter 801 is a range between any two diameters listed above.

The stop member 800,800′ of various embodiments may include natural materials, synthetic materials, metals, plastics, or carbon-based materials. The materials of various embodiments are materials acceptable for use in organisms such as human being or mammals or are biocompatible materials. Examples of such materials can include polyolefins such as polypropylenes and polyethylenes; polyamides such as nylons; polyesters such as polyethylene terephthalate; polymethylmethacrylate; styrene acrylonitrate; cellulose esters such as cellulose propionate; block copolymers include styrenes (for example styrene ethylene butadiene styrene, or styrene butadiene styrene), polyolefins (for example polypropylene/ethylene propylene diamine modified systems (i.e. synthetic rubber)), polyamides (for example polyamide (2 or polyamide 6), polyesters (for example polyester ester or polyether ester), polyurethanes (for example polyesterurethane, polyetherurethane or polyesteretherurethane); polyketones (e.g., polyether ether ketones (PEEK), polyaryl ether ketones (PAEK) or mixtures thereof), polyphenylene sulfide (PPS); natural plant-based fibers; or metals such as titanium, aluminum, brass, steel, carbon steel, or stainless steel.

In various embodiments are disclosed kits for use with a catheter including: an elongated flexible guidewire including an end portion resiliently biased in a curved configuration, the end portion having a first section including a distal end of the guidewire and a second section adjacent to the first section, and an engaging member attached to a portion of the first section adjacent to the distal end; and a container with an interior containing the guidewire. In various embodiments, the kits can further include a torque device of any embodiment or cap(s) capable of removably sealing the ports of the catheter. The kits of various embodiments can also include and adapter with an elongated body having a lumen capable of receiving the guidewire, an end portion sized or shaped for attachment to a port of the catheter, and a hemostasis valve disposed within the lumen. In various embodiments, the adapter has a second valve fluidly connected to the lumen of the elongated body.

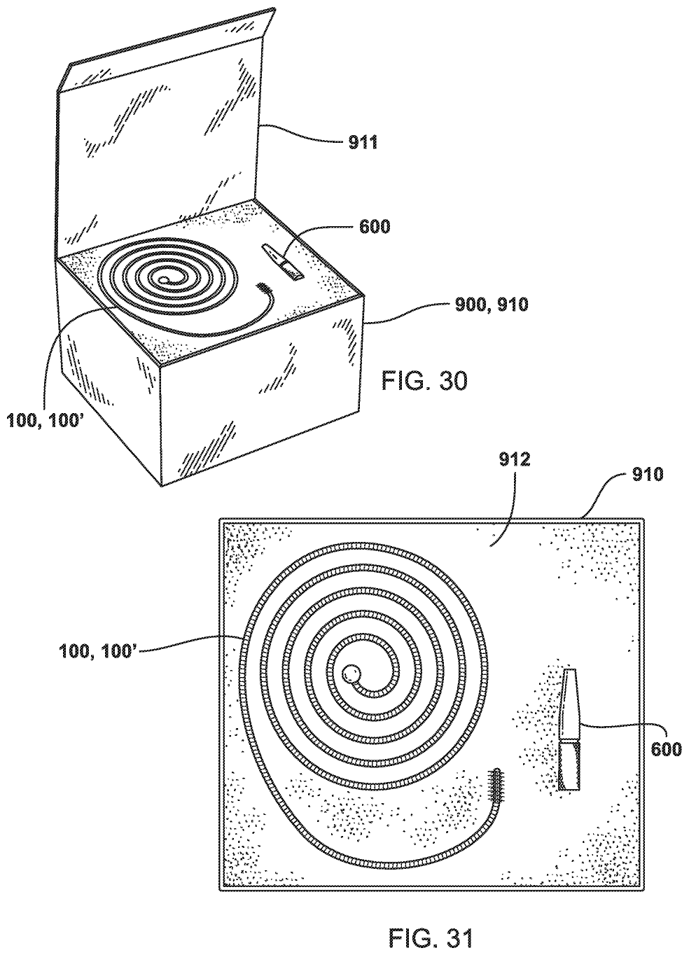

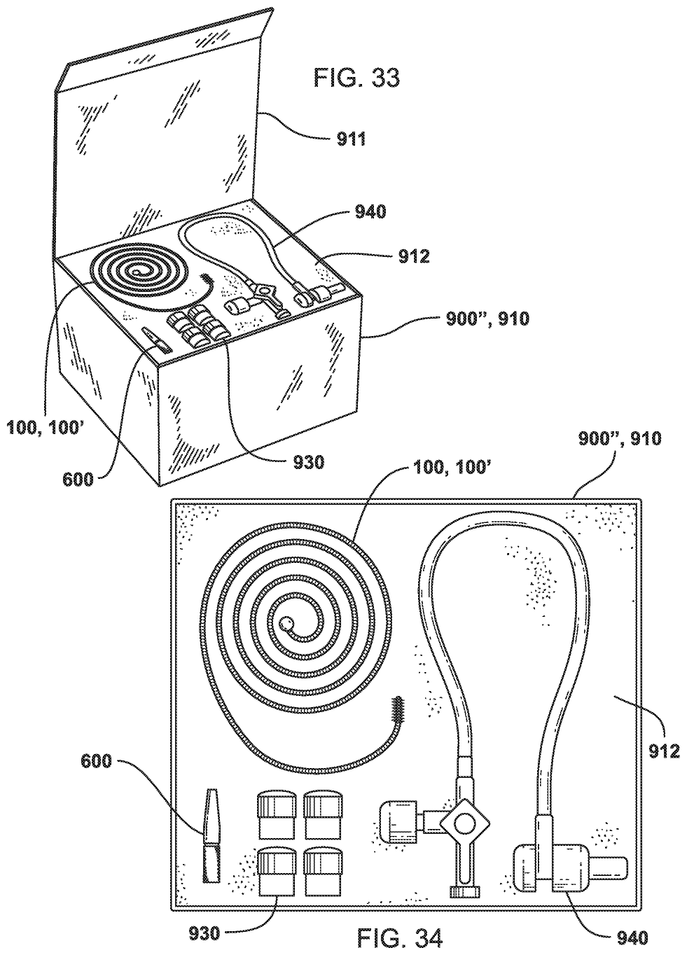

FIGS. 30, 31, 32, 33, and 34 are views showing kits 900,900′,900″ of various embodiments including a container 910 or packaging 920. FIGS. 30 and 31 show a kit 900 having a guidewire 100,100′ and torque device 600 contained within a container 910. The container 910 can further include a lid 911 to removably cover the guidewire 100,100′ and torque device 600 and padding 912 in which the guidewire 100,100′ and torque device 600 can be placed within. FIGS. 33 and 34 further shows a kit 900″ having a guidewire 100,100′, torque device 600, cap 930, and a hemostasis adapter 940 contained within the container 910.

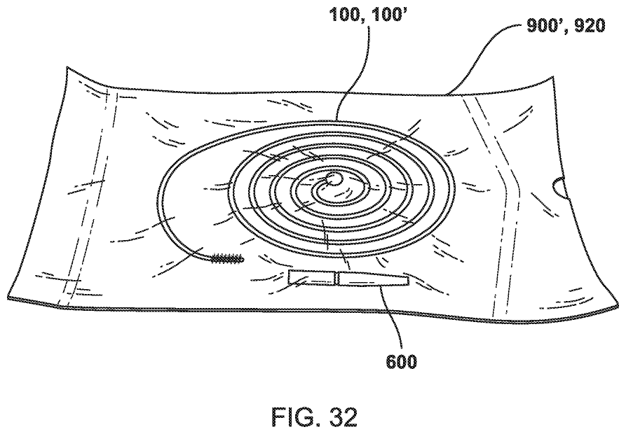

FIG. 32 shows a kit 900′ having a guidewire 100,100′ and torque device 600 contained within packaging 920. The packaging 920 can be autoclavable to allow for sterilization of the guidewire 100,100′ and torque device 600. The packaging 920 can also include a cap 930 or a hemostasis adapter 940.

In various embodiments are disclosed methods of conditioning surfaces of a catheter including: providing an elongated flexible guidewire including an end portion resiliently biased in a curved configuration, the end portion having a first section including a distal end of the guidewire and a second section adjacent to the first section, and an engaging member attached to a portion of the first section adjacent to the distal end; inserting the guidewire into a proximal port of a catheter inserted into a blood vessel, where the proximal port is fluidly connected to a lumen defined by a wall and a distal port; and conditioning a surface of the wall by advancing the guidewire through the lumen, where the engaging member contacts the wall.

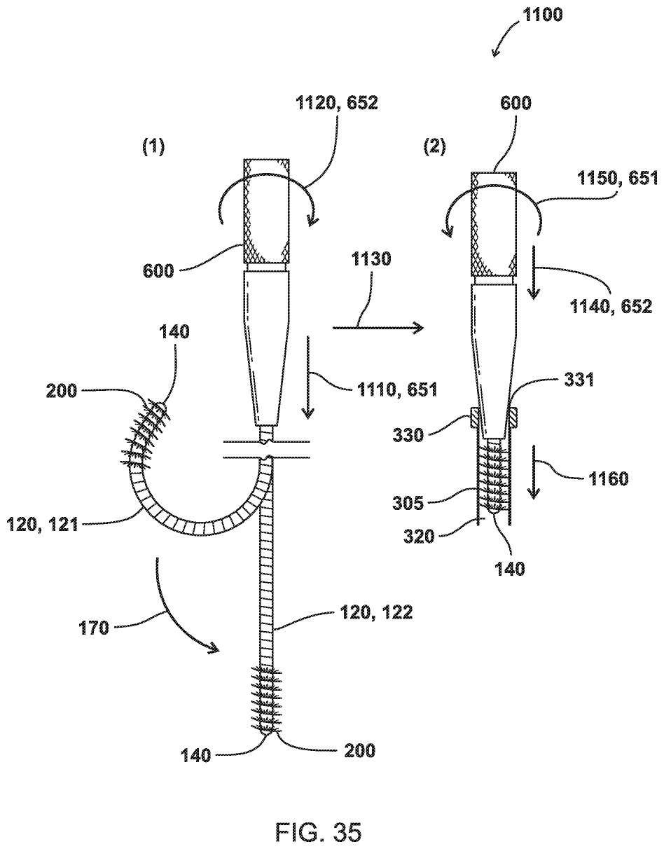

FIG. 35 shows views of methods 1100 for using the systems/devices 500,500′,500″,500″′ of various embodiments in which the end portion 120 of the flexible guidewire 100,100′ is transitioned to the straightened configuration 122 and inserted into an opening 331 of a port 330 of a catheter 300.

As shown in FIG. 2 and (1) of FIG. 35, the end portion 120 of the flexible guidewire 100,100′ is transitioned 170 to the straightened configuration 122. As shown in (1) of FIG. 35, the torque device 600 in the first position 651 can be positioned 1110 along the flexible guidewire 100,100′ such that the end portion 120 is prevented from returning to the curved configuration 121. In this position, a user can transition 1120 the torque device 600 to the second position 652 to clamp the flexible guidewire 100,100′. Moving 1130 from (1) to (2) of FIG. 35, the torque device 600 in the second position 652 is inserted 1140 in the opening 331 of the port 330 such that the distal end 140 of the flexible guidewire 100,100′ is positioned past the opening 331 and optionally within the lumen 320. The torque device 600 is transitioned 1150 to the first position 651, where a user can slidably move 1160 the flexible guidewire 100,100′ within the lumen 320 of the catheter 300.

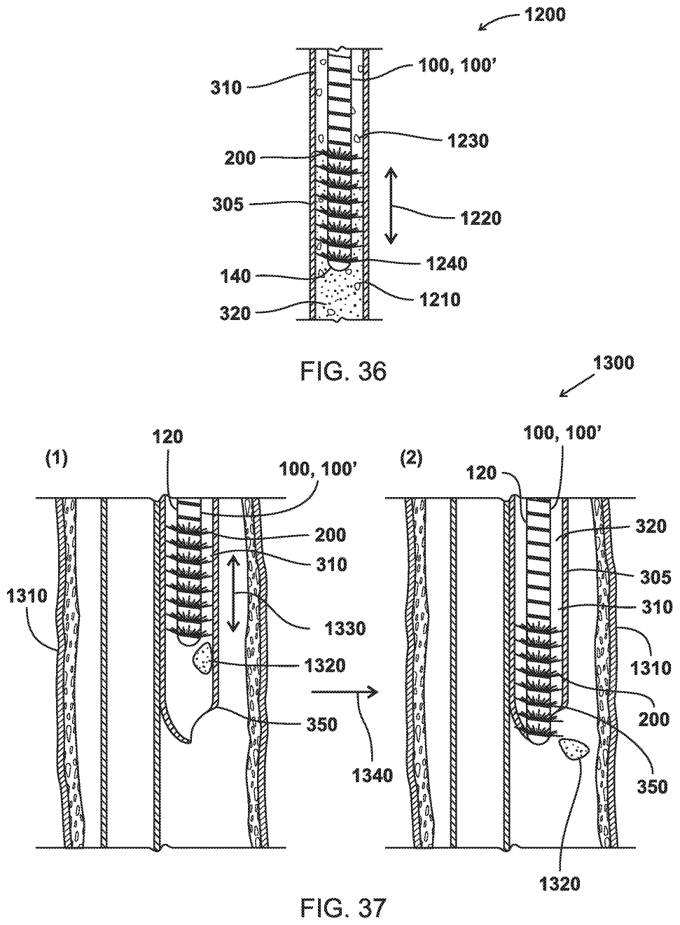

FIG. 36 shows a view of methods 1200 for using the systems/devices 500,500′,500″,500″′ of various embodiments in which the engaging member 200 contacts 1240 the inner surfaces 310 of the shaft 305 and dislodges debris 1210, 1230 such as cellular debris within the lumen 320 of the catheter 300. As shown in FIG. 36, debris 1210 can be deposited on the inner surfaces 310 of the shaft 305 and potentially block flow within the lumen 320. When the flexible guidewire 100,100′ is inserted into the lumen 320, the engaging member(s) 200 contacts 1240 the inner surfaces 310 of the shaft 305. A user can move 1220 the guidewire 100,100′ within the lumen 320 such that the engaging member(s) 200 dislodges the debris 1230 from the inner surfaces 310, which opens up flow through the lumen 320.

In various embodiments, the conditioning and extending include clearing an occlusion within the catheter. The clearing of various embodiments can include dislodges occlusions within a lumen of the catheter or removing the dislodged occlusions from the catheter.

FIG. 37 shows views of methods 1300 for using the systems/devices 500,500′,500″,500″′ of various embodiments in which the end portion 120 of the guidewire 100,100′ or engaging member 200 contacts an occlusion 1320 and dislodges the occlusion 1320 from the lumen 320 and into a blood vessel 1310. In one example, the occlusion 1320 is a thrombus occluding flow through the lumen 320. As shown in (1) of FIG. 37, a catheter 300 is positioned within a blood vessel, where shafts 305 can receive a fluid flow with in a blood vessel 1310 or direct fluid flow to the blood vessel 1310 through an opening 350. An occlusion 1320 is attached to the inner surface 310 of the shaft 305 and can block flow through the lumen 320. The guidewire 100,100′ can be moved 1320 through the lumen 320 to contact the occlusion 1320. As shown in (1) of FIG. 37, the end portion 120 or engaging member 200 can contact and dislodge the occlusion 1320 from the inner surface. Moving 1340 from (1) to (2) of FIG. 37, the end portion 120 or engaging member 200 can dislodge the occlusion 1320 from the lumen 320 and into the blood vessel 1310 through the opening 350.

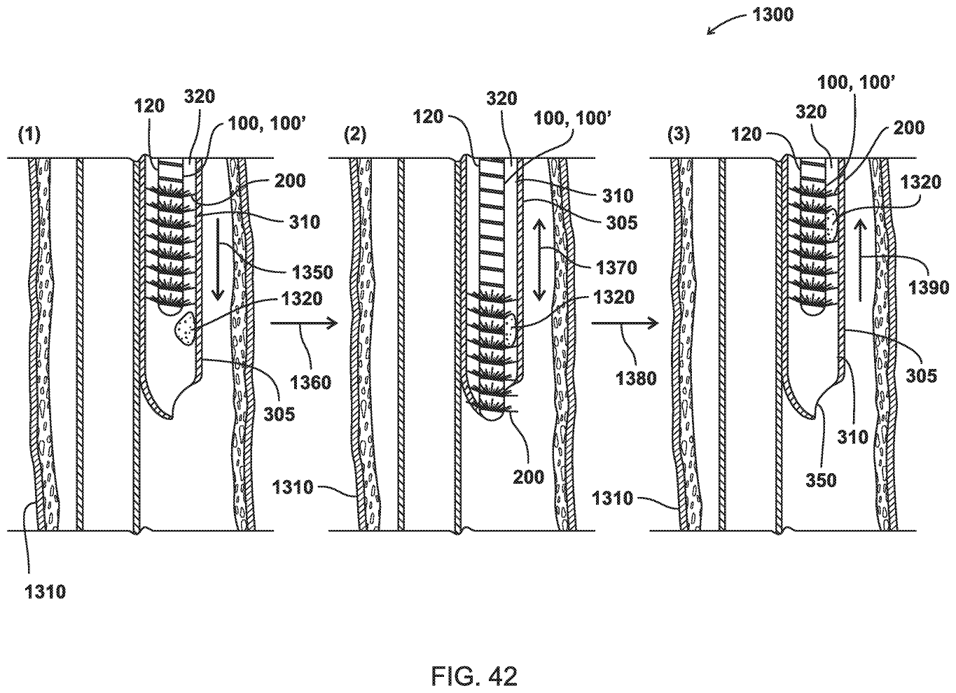

FIG. 42 shows views of methods 1300′ for using the systems/devices 500,500′,500″,500″′ of various embodiments in which the end portion 120 of the guidewire 100,100′ or engaging member 200 contacts an occlusion 1320 and clears the occlusion 1320 from the lumen 320 of the catheter 300. In one example, the occlusion 1320 is a thrombus occluding flow through the lumen 320. Similar to (1) of FIG. 37, (1) of FIG. 42 shows a catheter 300 positioned within a blood vessel, where shafts 305 can receive a fluid flow with in a blood vessel 1310 or direct fluid flow to the blood vessel 1310 through an opening 350. An occlusion 1320 is attached to the inner surface 310 of the shaft 305 and can block flow through the lumen 320. The guidewire 100,100′ is moved 1350 through the lumen 320 to contact the occlusion 1320. Moving 1360 from (1) to (2) of FIG. 42, the end portion 120 or engaging member 200 can contact and dislodge the occlusion 1320 from the inner surface. The guidewire 100,100′ can be moved 1370 in a manner such that the end portion 120 or engaging member 200 can dislodge the occlusion 1320 from the lumen 320. For example as shown in (2) of FIG. 42, guidewire 100,100′ is axially moved back and forth 1370 within the lumen 320 such that the end portion 120 or engaging member 200 dislodges the occlusion 1320 from the lumen 320. Further, the dislodged occlusion 1320 can become attached to the guidewire 100,100′ or entrapped by the end portion 120 or engaging member 200 such that the occlusion 1320 can be removed from the catheter 300 by removing the guidewire 100,100′ from the catheter 300. Moving 1380 from (2) to (3) of FIG. 42, the occlusion 1320 is attached or entrapped to the guidewire 100,100′ and the guidewire 100,100′ is axially moved 1390 away from the opening 350. Alternatively, the end portion 120 or engaging member 200 can obstruct the occlusion 1320 from entering the blood vessel 1310. When the guidewire 100,100′ is axially moved 1390, the dislodged occlusion 1320 moves with the guidewire 100,100′. The dislodged occlusion 1320 can move with the guidewire 100,100′ through the lumen 320 of the catheter 300 and eventually pass the opening 331 of the port 330.

In various embodiments, the methods of conditioning surfaces of a catheter can further include extending the engaging member past the distal port into the blood vessel, wherein the end portion biases towards the curved configuration. In various embodiments, the extending includes contacting the engaging member to an outer surface of the catheter to remove fibrin deposited on the outer surface.

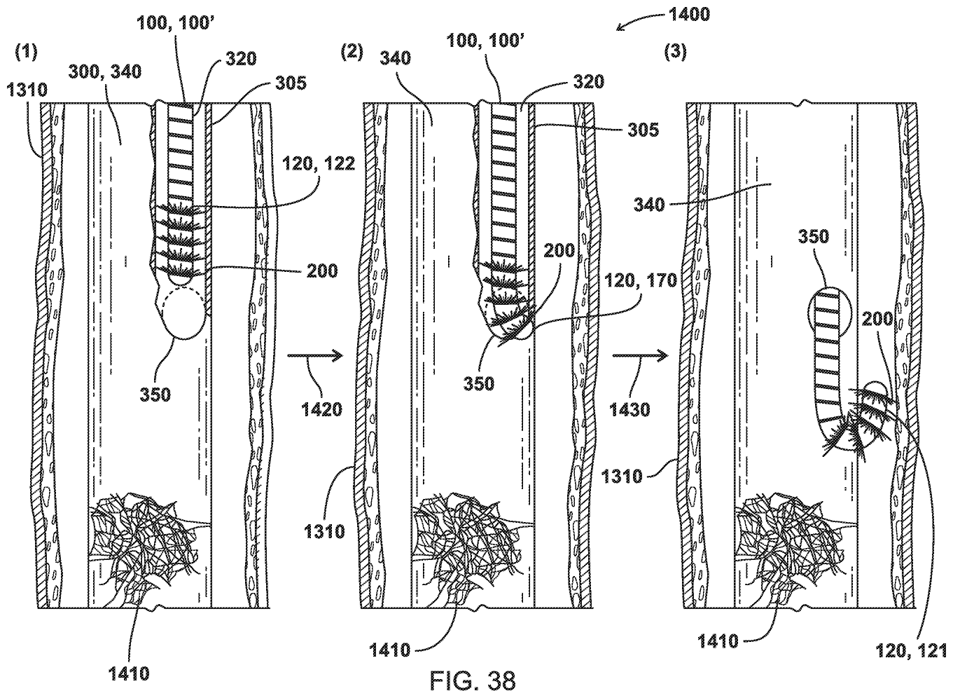

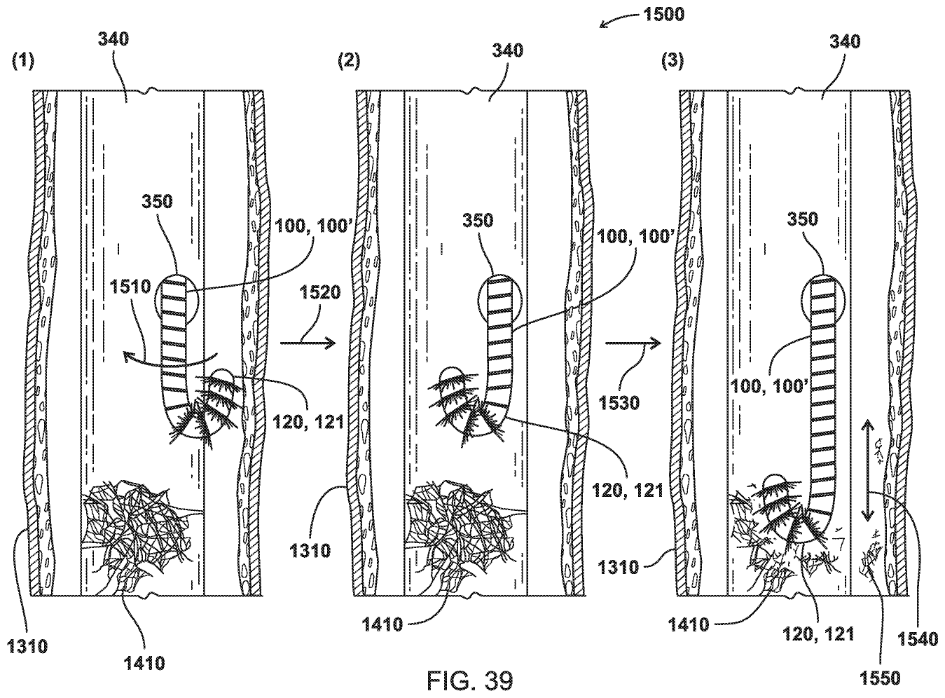

FIG. 38 shows views of methods 1400 for using the systems/devices 500,500′,500″,500″′ of various embodiments in which a user extends the engaging member 200 past a distal port/opening 350 of the catheter 300 and into the blood vessel 1310, where the end portion 120 biases towards the curved configuration 121. FIG. 39 shows views of methods 1500 for using the systems/devices 500,500′,500″,500″′ of various embodiments in which the flexible guidewire 100,100′ extending through the distal port 350 of the catheter 300 is moved 1540 to contact and remove debris/film 1410 attached to the outer surface 340 of the catheter 300.

As shown in (1) of FIG. 38, a catheter 300 is positioned within a blood vessel 1310, where shafts 305 can receive a fluid flow with in a blood vessel 1310 or direct fluid flow to the blood vessel 1310 through an opening 350. A film or deposit 1410 such as fibrin coating/film is attached to the outer surface 340 of the catheter 300. The flexible guidewire 100,100′ can be used to remove the deposit 1410. Moving 1420 from (1) to (2) of FIG. 38, a user extends the flexible guidewire 100,100′ past the opening 350, where the end portion 120 transitions 170 to the curved configuration 121. Moving 1430 from (2) to (3) of FIG. 38, the end portion 120 is in the curved configuration 121 when the engaging member 200 extends past the opening 350 into the blood vessel 1310.

As shown in (1) of FIG. 39, the flexible guidewire 100,100′ can be rotated 1510, for example, by the torque device 600 in the second position 652. Moving 1520 from (1) to (2) of FIG. 39, the rotation 1510 positions the flexible guidewire 100,100′ such that the end portion 120 or engaging member 200 contacts the outer surface 340 of the catheter 300. Moving 1530 from (2) to (3) of FIG. 39, a user can extend 1540 the flexible guidewire 100,100′ along the outer surface 340 such that the end portion 120 or engaging member 200 contacts the debris/film 1410. The user can then operate 1540 the flexible guidewire 100,100′ via the end portion 120 or engaging member 200 to dislodge 1550 the debris/film 1410 from the outer surface 340 of the catheter 300.

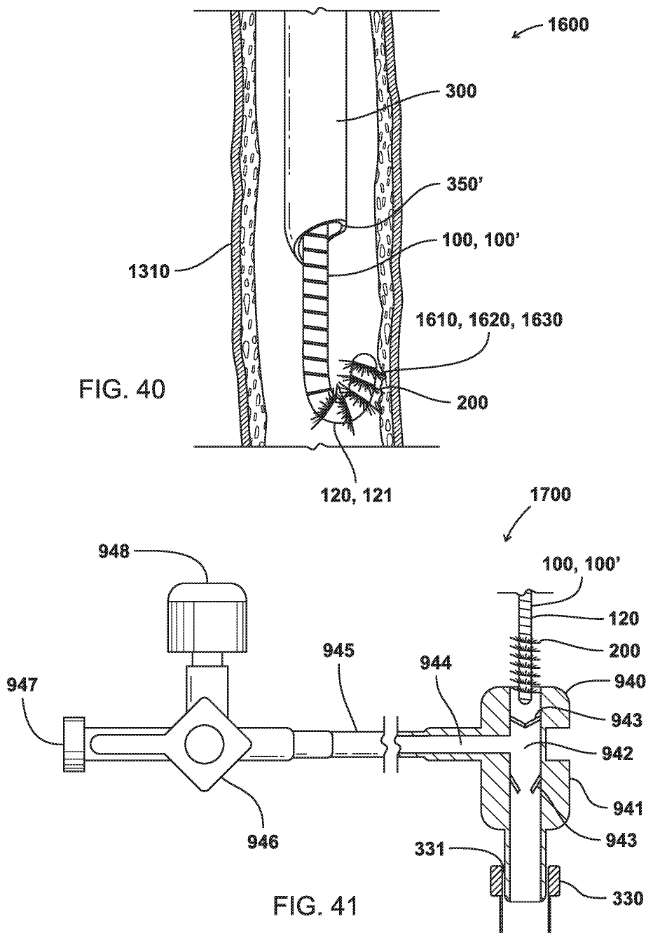

In various embodiments, the extending includes collecting tissue or cell samples with the engaging member. FIG. 40 shows a view of methods 1600 for using the systems/devices 500,500′,500″,500″′ of various embodiments in which the flexible guidewire 100,100′ extending through the distal port 350′ of the catheter 300 contacts 1610 and samples 1630 cells or tissues 1620 within a blood vessel 1310. As shown in FIG. 40, a user moves the flexible guidewire 100,100′ through the lumen of the catheter 300 such that the end portion 120 and engaging member 200 extends past the opening 350′, where the end portion 120 in the curved configuration 121 or the engaging member 200 contacts 1620 cells or tissues within a blood vessel 1310. The end portion 120 or engaging member 200 can entrap 1630 cells or tissues, which can be used as samples for further testing or analysis.

In various embodiments, the methods can further include attaching an adapter with an elongated body having a lumen capable of receiving the guidewire, an end portion sized or shaped for attachment to a port of the catheter, and a hemostasis valve disposed within the lumen to the port such that the lumens of the adapter and catheter are connected; and inserting the flexible guidewire through the lumen of the adapter and into the opening of the port.

The adapter of various embodiments can further include a port connected to the lumen and capable of receiving fluids from the catheter or cells and tissues entrapped within the end portion or engaging member when the end portion is positioned past the hemostasis valve. In various embodiments, the methods include drawing a cell or tissue sample from the end portion positioned past the hemostasis valve by drawing fluids from the catheter.

FIG. 41 shows a view of methods 1700 for using the systems/devices 500,500′,500″,500″′ of various embodiments in which an adapter 940 is attached to the port 330 of the catheter 300 and the flexible guidewire 100,100′ is insert through the adapter 940 and into the opening 331 of the port 300. The hemostasis adapter 940 includes elongated body 941 having a lumen 942. The elongated body 941 is capable of attachment to the port 330 of a catheter 300 by various attachments including, for example, swivel nut luer fittings. When attached, the lumen 942 of the elongated body 941 is connected to the lumen 320 of the catheter 300. A plurality of spaced apart hemostasis valves 943 are disposed within the lumen 942. A port 944 positioned between the hemostasis valves 943 is connected to the lumen. The port 944 is fluidly connected (i.e. by tubing 945) to a valve 946 having ports 947,948. A user can attach a syringe to the ports 947,948 and engage the valve 946 to direct flow to the ports 947,948 to draw fluid from the catheter or cells/tissues from the guidewire 100,100′ (not shown). As shown in FIG. 41, the method 1700 includes attaching the hemostasis adapter 940 to the port 330 of the catheter 300 and inserting the guidewire 100,100′ through the lumen 9442 and into the opening 331 of the port 330. The method 1700 can further include drawing fluid from the catheter 300; through the lumen 942, side port 944, and tubing 945; and to the port 947,948. The method 1700 can further include acquiring cells or tissues from the engaging member 200 by positioning the end portion 120 or engaging member 200 between the hemostasis valves 943; and drawing fluid from the catheter 300 as previously described; wherein the fluid dislodges the cells or tissues from the engaging member 200.

In various embodiments, a user aspirates the hemostasis adapter and catheter with the syringe to draw an occlusion attached or entrapped to the guidewire. As previously stated, the dislodged occlusion 1320 can move with the guidewire 100,100′ through the lumen 320 of the catheter 300 and eventually passed the opening 331 of the port. When the end portion 120 is positioned between the hemostasis valves 943, a user can aspirate the hemostasis adapter 940 the draw the dislodged occlusion 1320 from the guidewire 100,100′; through the side port 944, tubing 945, and ports 947,948; and into a cavity of the syringe. In alternative embodiments, the occlusion can be extracted from the catheter via aspiration with the syringe after the occlusion is dislodged with the guide wire. The end portion or engaging member can obstruct the occlusion from entering the blood vessel.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for use with a catheter comprising:
an elongated flexible guidewire capable of being slidably received within the catheter and including an end portion resiliently biased in a curved configuration, the end portion having a first section including a distal end of the guidewire and a second section adjacent to the first section, and an engaging member attached to a portion of the first section adjacent to the distal end.

2. The system of claim 1, wherein the catheter is a dialysis catheter.

3. The system of claim 1, wherein the first section has a length less than a length of the second section.

4. The system of claim 1, wherein the engaging member includes a plurality of bristles attached to the portion of the first section.

5. The system of claim 4, wherein the plurality of bristles are attached to the portion of the first section at angles of 90 degrees or more relative to an outer surface of the portion of the first section and the distal end in the curved configuration.

6. The system of claim 1, wherein the engaging member includes a mesh substrate attached to the portion of the first section.

7. The system of claim 1, wherein the engaging member includes a fin attached to the portion of the first section.

8. The system of claim 1, wherein the guidewire has a plurality of evenly spaced indicia visible to identify measured distances from the distal end.

9. The system of claim 1 further comprising a stop member attached to the guidewire and configured to obstruct a portion of the guidewire from entering the catheter.

10. The system of claim 9, wherein the portion of the guidewire from entering the catheter includes a proximal end or the distal end of the guidewire.

11. The system of claim 1 further comprising:
a torque device including
an elongated body having a lumen capable of receiving the guidewire and an end portion sized or shaped for insertion into a port of the catheter;
an actuator operably connected to the elongated body and having a lumen configured for selective engagement of the guidewire, where the actuator is movable between a position where the guidewire is secured relative to the torque device and a position where the guidewire can movable relative to the torque device.

12. The system of claim 11 further comprising a stop member attached to the guidewire and configured to obstruct a portion of the guidewire from entering the lumen of the elongated body.

13. The system of claim 12, wherein the portion of the guidewire from the lumen of the elongated body includes a proximal end or the distal end of the guidewire.

14. A kit for use with a catheter comprising:
an elongated flexible guidewire including an end portion resiliently biased in a curved configuration, the end portion having a first section including a distal end of the guidewire and a second section adjacent to the first section, and an engaging member attached to a portion of the first section adjacent to the distal end; and
a container with an interior containing the guidewire.

15. The kit of claim 14 further comprising:
a torque device within the interior of the container, the torque device including
an elongated body having a lumen capable of receiving the guidewire and an end portion sized or shaped for insertion into a port of the catheter; and
an actuator operably connected to the elongated body and having a lumen configured for selective engagement of the guidewire, where the actuator is movable between a position where the guidewire is secured relative to the torque device and a position where the guidewire can movable relative to the torque device.

16. The kit of claim 14 further comprising an adapter within the interior of the container, the adapter including an elongated body having a lumen capable of receiving the guidewire, an end portion sized or shaped for attachment to a port of the catheter, and a hemostasis valve disposed within the lumen.

17. A method of conditioning surfaces of a catheter comprising:
providing an elongated flexible guidewire including an end portion resiliently biased in a curved configuration, the end portion having a first section including a distal end of the guidewire and a second section adjacent to the first section, and an engaging member attached to a portion of the first section adjacent to the distal end;
inserting the guidewire into a proximal port of a catheter inserted into a blood vessel, where the proximal port is fluidly connected to a lumen defined by a wall and a distal port;
conditioning a surface of the wall by advancing the guidewire through the lumen, where the engaging member contacts the wall; and
extending the engaging member past the distal port into the blood vessel, wherein the end portion biases towards the curved configuration.

18. The method of claim 17, wherein the conditioning and extending include clearing an occlusion within the catheter.

19. The method of claim 17, wherein the extending includes contacting the engaging member to an outer surface of the catheter to remove deposits on the outer surface.

20. The method of claim 17, wherein the extending includes collecting tissue or cell samples with the engaging member.
